(19) United States
(12) Patent Application Publication
(10) Pub. No.: US 2010/0145234 A1
(43) Pub. Date: Jun. 10, 2010

(54) HAND-HELD DEVICE FOR DETECTING ACTIVITIES OF DAILY LIVING AND SYSTEM HAVING THE SAME

(76) Inventors: Jae Won JANG, Daejeon (KR); Sa Kwang SONG, Daejeon (KR); Ho Youl JUNG, Daejeon (KR); Min Ho KIM, Daejeon (KR); Soo Jun PARK, Seoul (KR); Seon Hee PARK, Daejeon (KR)

Correspondence Address: LADAS & PARRY LLP, 224 SOUTH MICHIGAN AVENUE, SUITE 1600, CHICAGO, IL, 60604, US

(21) Appl. No.: 12/491,304

(22) Filed: Jun. 25, 2009

(30) Foreign Application Priority Data
Dec. 8, 2008 (KR) 10-2008-0124282

(51) Int. Cl. A61B 5/11

(52) U.S. Cl. 600/595

(57) ABSTRACT

There are provided a hand-held device for detecting activities of daily living, and a system for detecting activities of daily living using the same. The hand-held device comprises a behavior information detector unit detecting physical activity information from user's movements; an article use information detector unit detecting article use information on whether the user uses the article from a sensor module attached to the article; a controller unit generating information on activities of daily living of the user from the physical activity information and the article use information; and a memory unit storing the information on the activities of daily living of the user.

### CROSS-REFERENCE TO RELATED APPLICATIONS

This application claims the priority of Korean Patent Application No. 2008-124282 filed on Dec. 8, 2008, in the Korean Intellectual Property Office, the disclosure of which is incorporated herein by reference.

### BACKGROUND OF THE INVENTION

1. Field of the Invention

The present invention relates to a hand-held device for detecting activities of daily living, and more particularly, to a hand-held device for detecting activities of daily living of individuals such as the elderly who live alone (hereinafter, referred to as “solitary elderly”), and a system for detecting activities of daily living using the same.

2. Description of the Related Art

Recent society has rapidly entered into an aging society. As a result, the elderly who live alone has been increased in number. In particular, the aged should necessarily receive health care due to their uneasy behavior and chronics diseases, and should be in continuous care due to the reduction in recognition abilities. However, the problems associated with the aged have developed into a social problem due to the manpower shortage and the increase in care costs. In order to solve the above problems, devices that can check the health of the solitary elderly in a remote-control manner have been developed. For example, devices in which IT technology is combined with sensor technology such as u-health care and smart home have been developed. The conventional technologies have focused on periodically monitoring a variety of biometric information (for example, blood pressure, glycosuria and the like) on the solitary elderly, etc. In addition, there are lots of active attempts on living cares to find anomaly symptoms of the solitary elderly and induce their healthy lives through the analysis of living patterns of the solitary elderly.

In the case of the conventional daily life monitoring technologies, a variety of sensors (for example, PIR, a touch sensor, a pressure sensor, an acceleration sensor, a heat sensor, a light sensor, an acoustic sensor, etc.) and devices (for example, CCTV, RFID, wire/wireless communication equipment) may be installed in places of residence to analyze information on a series of life changes and states according to living patterns of the residents, thus to detect activities of daily living of the residents.

However, due to the difference in living environments and living patterns of individuals, a variety of sensors should be used to solve these problems. Therefore, the use of the various sensors leads to more complicated systems and cause much inconvenience to the residents.

### SUMMARY OF THE INVENTION

The present invention is designed to solve the problems of the prior art, and therefore it is an object of the present invention to provide a hand-held device and system that may more accurately detect activities of daily living of users such as residents (particularly, the solitary elderly) even though the minimum number of sensors are provided in places of residence.

According to an aspect of the present invention, there is provided a hand-held device for detecting activities of daily living. Here, the hand-held device includes a behavior information detector unit detecting physical activity information from user's movements; an article use information detector unit detecting article use information on whether the user uses the article from a sensor module attached to the article; a controller unit generating information on activities of daily living of the user from the physical activity information and the article use information; and a memory unit storing the information on the activities of daily living of the user.

In this case, the behavior information detector unit may include one selected from the consisting of an acceleration sensor and a gyroscope.

Also, the hand-held device may further include a clock unit providing time information, and the controller unit may generate information on the activities of daily living of the user according to the time information provided from the clock unit.

In addition, the controller unit may generate information on the activities of daily living of the user in a predetermined cycle, and also may generate information on the activities of daily living of the user by synchronizing the physical activity information and the article use information.

Meanwhile, the behavior information detector unit detects the physical activity information with reference to the data on physical activities corresponding to the user's physical activities stored in the memory unit.

According to another aspect of the present invention, there is provided a system for detecting activities of daily living of users. Here, the system includes a sensor module attached to an article to provide information on use of articles with a user; a hand-held device for detecting activities of daily living, comprising a behavior information detector unit detecting physical activity information from user's movements, an article use information detector unit detecting article use information on whether the user uses the article from the information on use of the articles, a controller unit generating information on activities of daily living of the user from the physical activity information and the article use information and a memory unit storing the information on the activities of daily living of the user; and a remote control device receiving the information on the activities of daily living from the hand-held device for detecting activities of daily living in order to take necessary measures against the user according to the information on the activities of daily living.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a schematic block diagram illustrating a hand-held device and system for detecting activities of daily living according to one exemplary embodiment of the present invention.

FIG. 2 is a block diagram illustrating a hand-held device for detecting activities of daily living according to one exemplary embodiment of the present invention.

FIG. 3 is a flowchart illustrating a method for detecting activities of daily living using the hand-held device and system according to one exemplary embodiment of the present invention.

### DETAILED DESCRIPTION OF THE PREFERRED EMBODIMENT

Exemplary embodiments of the present invention will now be described in detail with reference to the accompanying drawings. In the accompanying drawings, the same or similar components have the same or similar reference numerals throughout the specification of the present invention.

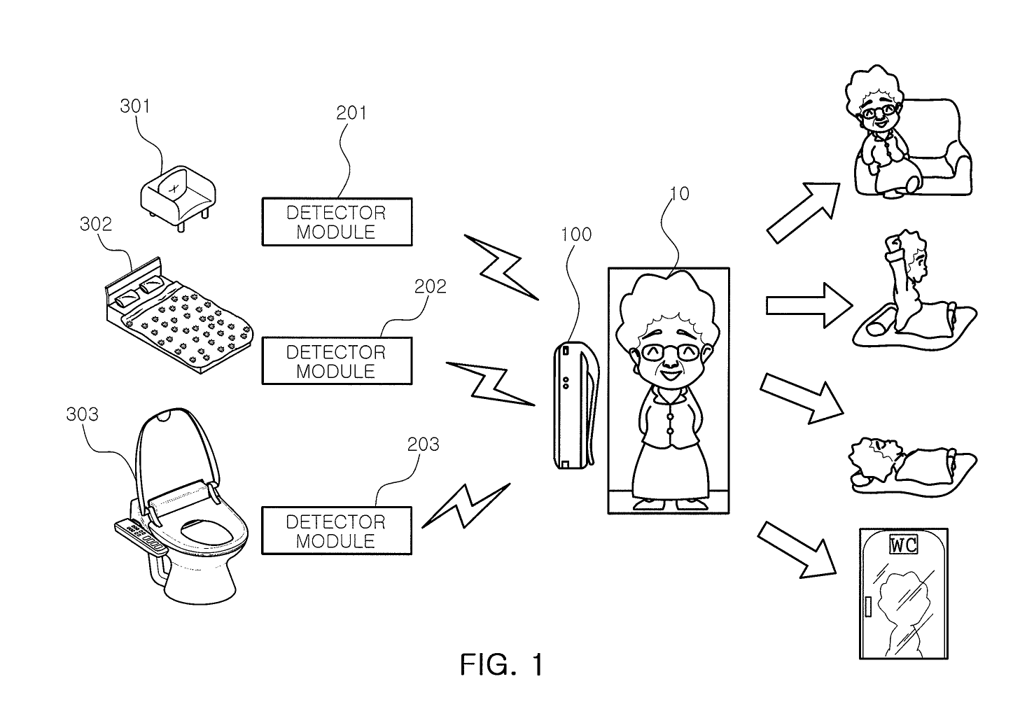

FIG. 1 shows one example of a hand-held device 100 for detecting activities of daily living according to one exemplary embodiment of the present invention. As shown in FIG. 1, the hand-held device 100 for detecting activities of daily living according to one exemplary embodiment of the present invention is carried by a user 10, and may detect activities of daily living of the user 10, such as states where the user 10 is sitting on a sofa, getting up, sleeping and going to stool, by receiving signals for the use of articles 301 to 303 (for example, as a sofa, a bed, a chamber pot and the like), which are equipped in a space of residence and the like, from sensor modules 201 to 203 attached to the articles 301 to 303. Along with the sensor modules 201 to 203, the hand-held device 100 for detecting activities of daily living according to one exemplary embodiment of the present invention may construct a system for detecting activities of daily living according to exemplary embodiment of the present invention, as shown in FIG. 1.

In addition to the sofa, the bed and the chamber pot as shown in FIG. 1, the articles 301 to 303 equipped in the space of residence and the like may further include articles such as furniture or electronic devices that are equipped in the space of residence and the like. The sensor modules 201 to 203 are provided with any of sensors that may sense the use of articles (for example, a pressure-detection sensor, a vibration-detection sensor and the like, which are installed in each of the article 301 to 303), thus to generate article use information on whether a user 10 uses the article. The sensor modules 201 to 203 are preferably attached to the articles 301 to 303 so that the user 10 cannot recognize the installation of the sensor modules 201 to 203. In this case, since the user 10 cannot visually sense the presence of the sensor modules 201 to 203, the user 10 can get rid of feelings of psychological anxiety and rejection that the user 10 is placed under surveillance.

The generated information is transmitted to the hand-held device 100 for detecting activities of daily living using a wireless communication system that includes, but is not particularly limited to, ZigBee, Bluetooth and USN. For example, the number of the article 301 to 303 as shown in FIG. 1 is 3, but the present invention is not particularly limited thereto. More than or less than 3 articles may also be installed in the place of residence. Therefore, the sensor modules may also be increased or decreased in number according to the number of the articles.

Although not shown in FIG. 1, the system for detecting activities of daily living according to one exemplary embodiment of the present invention may further include a remote control device receiving the information on the activities of daily living of the user 100 from the hand-held device 100 for detecting activities of daily living and setting necessary measures for the user according to the information on the activities of daily living.

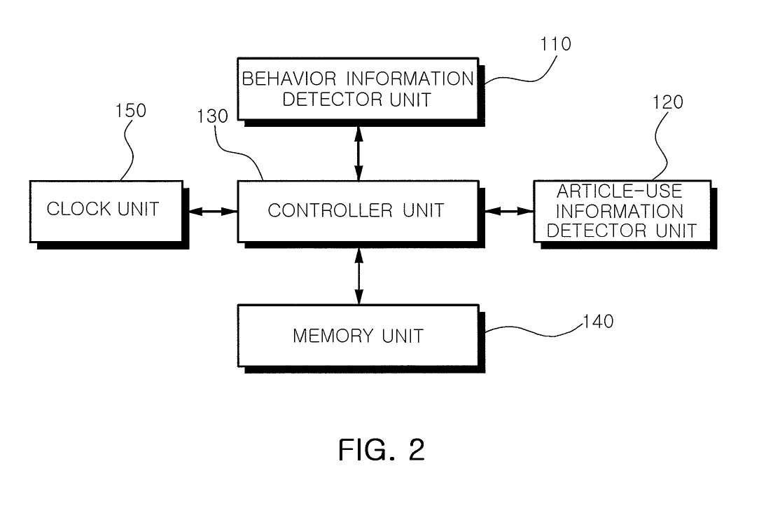

FIG. 2 is a block diagram illustrating a hand-held device 100 for detecting activities of daily living according to one exemplary embodiment of the present invention. As shown in FIG. 2, the hand-held device 100 further include a behavior information detector unit 110 detecting information on physical activities of the user 10, an article use information detector unit 120 receiving article use information on whether the user 10 uses the articles 301 to 303 from the sensor modules 201 to 203 attached to the articles 301 to 303 that are installed in a place of residence, as shown in FIG. 1, and detecting the article use information on whether the user 10 uses the articles 301 to 303, a controller unit 130 generating information on the activities of daily living of the user 10 from the physical activity information of the user 10 detected in the behavior information detector unit 110 and the article use information detected in the article use information detector unit 120, and a memory unit 140 storing the information on the activities of daily living. In addition, the hand-held device 100 according to one exemplary embodiment of the present invention further includes a clock unit providing time information, as shown in FIG. 2. In this case, the controller unit 130 may generate information on the activities of daily living of the user 10 by use of the time information provided from the clock unit 150, that is, time information including start time, duration time and end time of the physical activities (or intermediate activities).

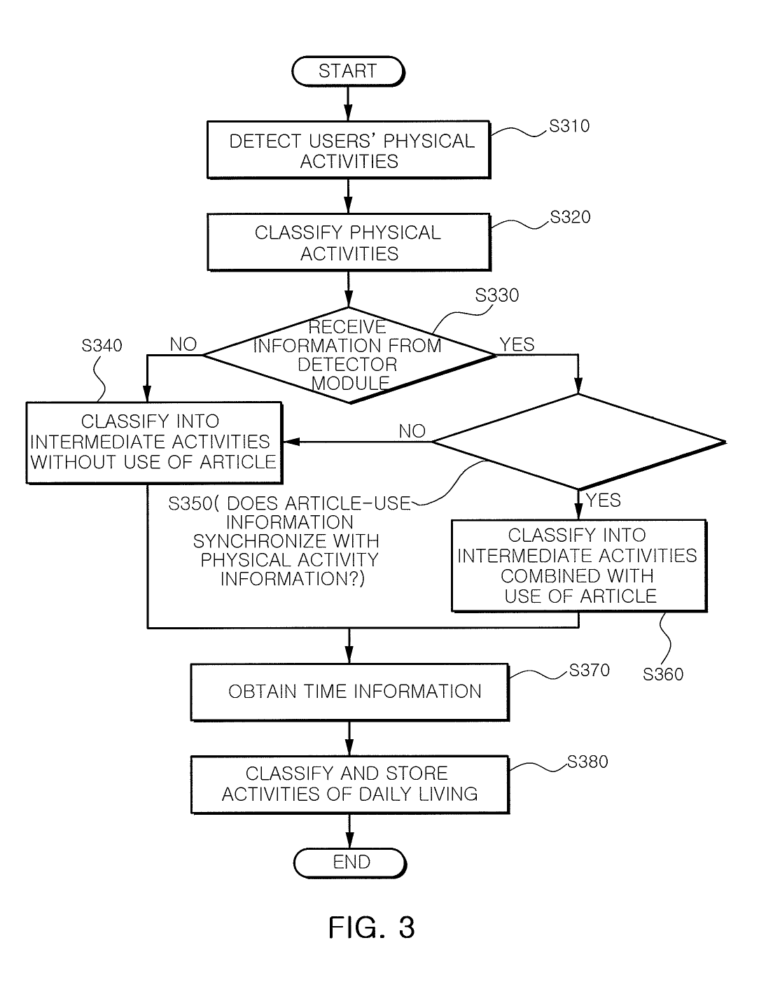

Hereinafter, the operations and functions of the hand-held device 100 and system for detecting activities of daily living according to one exemplary embodiment of the present invention are described in more detail with reference to FIGS. 1 to 3. FIG. 3 is a flowchart illustrating a method for detecting activities of daily living using the hand-held device 100 and system according to one exemplary embodiment of the present invention.

The hand-held device 100 for detecting activities of daily living according to one exemplary embodiment of the present invention is carried by the user 10 such as the solitary elderly. While the user 10 carries the hand-held device 100, the hand-held device 100 for detecting activities of daily living is used to detect the activities of daily living of a carrier, that is, a user 10. When the hand-held device 100 for detecting activities of daily living is turned on, the article use information detector unit 120 of the hand-held device 100 for detecting activities of daily living is in a signal-receiving standby state where the hand-held device 100 may communicate with the sensor modules 201 to 203. In this case, when the user 10 begins to move, the behavior information detector unit 110 detects the information on physical activities of the user 10 (S310 of FIG. 3). The behavior information detector unit 110 includes an acceleration sensor and a gyroscope, but the present invention is not particularly limited thereto. The behavior information detector unit 110 may include any of sensors that can detect the physical activities of the users (carriers). Meanwhile, the hand-held device 100 for detecting activities of daily living may include a power supply unit (not shown) such as a portable battery. Here, the hand-held device 100 preferably operates by power supply from the portable battery.

In general, a motion acceleration is generated according to the physical activities of the user 10. In this case, an acceleration sensor detects the generated motion acceleration to filter X-axis, Y-axis and Z-axis components of the motion acceleration and X-axis, Y-axis and Z-axis components of the gradient. Next, a value for each filtered component is converted into a vector value, and the converted vector value is used to classify the physical activities of the user 10 with reference to the data on the physical activities of the user stored in the memory unit 140 (S320 of FIG. 3). For example, data (for example, vector values) on the physical activities such as “sit,” “stand,” “lie,” and “move” are stored in the memory unit 140, and the acceleration sensor may use the converted vector values to classify the information on physical activities of the user 10 referring to the data.

The next operation is to determine whether the information on the use of the articles 301 to 303 provided with the sensor modules 201 to 203 is received from the sensor modules 201 to 203 (S330 of FIG. 3). The sensor module 201 to 203 may provide an On signal when the articles 301 to 303 are being used, and provide an Off signal when the articles 301 to 303 are not used. Instead, the sensor modules 201 to 203 may provide each of corresponding signals as the article use information when the state of the articles 301 to 303 is changed from the close state to the use state or the state of the articles 301 to 303 is changed from the use state to the close state.

As described above, the article use information detector unit 120 detects the article use information including information on the use of the articles 301 to 303 by receiving the article use information from the sensor modules 201 to 203, and the controller unit 130 classifies the physical activities of the user 10 into intermediate activities in which there is no article use information (S340) when, from the article use information, it is determined that there is none of the articles 301 to 303 that are being used (“NO” in S330 of FIG. 3).

On the contrary, when the articles 301 to 303 are being used (S330 of FIG. 3), the controller unit 130 inspects whether the physical activity information and the article use information synchronize with each other (S350 of FIG. 3). When the physical activity information and the article use information do not synchronize with each other, the controller unit 130 classifies the physical activities of the user 10 into intermediate activities where there is no article being used (S340). On the contrary, when both of the information synchronize with each other, the controller unit 130 classifies the physical activities of the user 10 into intermediate activities combined with the use of the articles (S360).

For example, when the physical activity of the user 10 is classified into “sit” as shown in S320 of FIG. 3, and the sensor module 201 attached to furniture (i.e., the sofa 301 of FIG. 1) generates a signal that is switched from “On” to “Off,” or generates a signal that continues to be in an On state, the physical activities such as “sit” or “sitting” on the sofa 301 may be recognized as the activities of daily living that are intermediate activities of the user who use the articles. Also, when the physical activity is “sit,” and there is a signal that the articles are not used, the information on the use of the furniture does not synchronize with the physical activities of the user 10. Therefore, the physical activities of the user 10 are classified into the intermediate activities where there is no article being used.

Subsequently, the controller unit 130 receives the time information on current time from the clock unit 150 (S370 of FIG. 3) to classify the detected physical activities of the user 10 and store the classified physical activities in the memory unit 140 (S380 of FIG. 3). That is, even when the classified physical activities of the user 10 have the same pattern, the corresponding activities of daily living of the user 10 may be varied with time. Therefore, the physical activities of the user 10 may be classified more accurately using the information on current time.

In this case, the classified intermediate activities may be recognized as the activities of daily living such as “taking a rest,” “getting up,” “going to stool” and “coming into action” using the time information such as “start time”, “duration time” and “end time”. For example, the difference between “going to bed” and “taking a rest” may be discriminated and recognized, as follows.

### EXAMPLE 1

### In the Case of the “Going to Bed”

A user lies in his bed 302 at ten o'clock.

In this case, the sensor module 202 transmits data on Bed “On” to the hand-held device 100 for detecting activities of daily living.

The hand-held device 100 for detecting activities of daily living classifies a physical activity of the user as “lying,” and synchronizes the received data on Bed “On.”

The hand-held device 100 classifies this intermediate activity into “lying in bed.”

The hand-held device 100 classifies the activity of daily living into “sleeping” using the time information.

### EXAMPLE 2

### In the Case of the “Taking a Rest”

A user lies in his bed 302 at eleven o'clock A.M.

In this case, the sensor module 202 transmits data on Bed “On” to the hand-held device 100 for detecting activities of daily living.

The hand-held device 100 for detecting activities of daily living classifies a physical activity of the user as “lying,” and synchronizes the received data on Bed “On.”

The hand-held device 100 classifies this intermediate activity into “lying in bed.”

The hand-held device 100 classifies the activity of daily living into “taking a rest” using the time information.

Meanwhile, the above-mentioned procedures may be used to classify the physical activities of the user in a predetermined cycle, for example, of 1 second. Here, the classified physical activities may be stored in the memory unit 140 for the future use. The stored data may be transmitted to other devices and equipment in order to deal with medical emergency, warning users, communicating with the authorities concerned.

Also, the information on the activities of daily living of the user 10 stored in the memory unit 140 of the hand-held device 100 for detecting activities of daily living may be transmitted to a remote control device for setting necessary measures for the user according to the information on the activities of daily living, thus to take a necessary measure for the user.

As described above, the hand-held device 100 for detecting activities of daily living according to one exemplary embodiment of the present invention may be useful to accurately and precisely recognize the activities of daily living of users using sensor modules for presenting the use of articles and hand-held devices for recognizing the use of the sensor modules. Also, the hand-held device 100 for detecting activities of daily living according to one exemplary embodiment of the present invention may be useful to get rid of feelings of psychological anxiety and rejection that the users are placed under surveillance since the user 10 cannot visually sense the presence of the sensor modules by simply installing the sensor modules in articles so that the sensor modules cannot be viewed by the users.

While the present invention has been shown and described in connection with the exemplary embodiments, it will be apparent to those skilled in the art that modifications and variations can be made without departing from the spirit and scope of the invention as defined by the appended claims.

What is claimed is:

1. A hand-held device for detecting activities of daily living, comprising:
a behavior information detector unit detecting physical activity information from user's movements;
an article use information detector unit detecting article use information on whether the user uses the article from a sensor module attached to the article;
a controller unit generating information on activities of daily living of the user from the physical activity information and the article use information; and
a memory unit storing the information on the activities of daily living of the user.

2. The hand-held device of claim 1, wherein the behavior information detector unit comprises one selected from the consisting of an acceleration sensor and a gyroscope.

3. The hand-held device of claim 1, further comprising a clock unit providing time information, and
wherein the controller unit generates information on the activities of daily living of the user according to the time information provided from the clock unit.

4. The hand-held device of claim 1, wherein the controller unit generates information on the activities of daily living of the user in a predetermined cycle.

5. The hand-held device of claim 1, wherein the controller unit generates information on the activities of daily living of the user by synchronizing the physical activity information and the article use information.

6. The hand-held device of claim 1, wherein the behavior information detector unit detects the physical activity information with reference to the data on physical activities corresponding to the user's physical activities stored in the memory unit.

7. A system for detecting activities of daily living, comprising:
a sensor module attached to an article to provide information on use of articles with a user;
a hand-held device for detecting activities of daily living, comprising a behavior information detector unit detecting physical activity information from user's movements, an article use information detector unit detecting article use information on whether the user uses the article from the information on use of the articles, a controller unit generating information on activities of daily living of the user from the physical activity information and the article use information, and a memory unit storing the information on the activities of daily living of the user; and
a remote control device receiving the information on the activities of daily living from the hand-held device for detecting activities of daily living and setting necessary measures for the user according to the information on the activities of daily living.
